(19) United States
(12) Patent Application Publication
(10) Pub. No.: US 2026/0370960 A1
(43) Pub. Date: Nov. 19, 2026

(54) OBSERVATION SYSTEM AND CONTROL DEVICE

(71) Applicant: Sony Olympus Medical Solutions Inc., Tokyo (JP)

(72) Inventors: Yoshiaki NIWA, Tokyo (JP); Hiroshi USHIRODA, Tokyo (JP)

(73) Assignee: Sony Olympus Medical Solutions Inc., Tokyo (JP)

(21) Appl. No.: 19/133,741

(22) PCT Filed: Nov. 14, 2023
(86) PCT No.: PCT/JP2023/041005

§ 371 Date: May 29, 2025

(30) Foreign Application Priority Data
Dec. 5, 2022 (JP) 2022-194241

(51) Int. Cl. H04N 23/88; A61B 1/00; A61B 1/04; A61B 1/06; G02B 21/00; G02B 21/06; G02B 21/36; H04N 23/74

(52) U.S. Cl. CPC H04N 23/88; A61B 1/00009; A61B 1/00045; A61B 1/0638; A61B 1/0655; G02B 21/0016; G02B 21/06; G02B 21/365; H04N 23/74; A61B 1/042

(57) ABSTRACT

An observation system includes a plurality of light sources, an imaging device, and a control device. The light sources apply light in different wavelength bands to a subject. The imaging device images the subject to which illumination light is applied. The illumination light includes beams of light from the plurality of light sources. The control device controls the operation of at least one of the plurality of light sources, and generates an output image to be output to the display device by performing image processing on a pixel signal acquired from the imaging device. The control device includes a light source control unit and an image processing unit. The light source control unit controls the operation of at least one of the plurality of light sources, and adjusts the color tone of the illumination light to a first color tone.

### FIELD

The present disclosure relates to an observation system and a control device.

### BACKGROUND

In the medical field and the industrial field, there is known an observation system for observing the inside of a subject such as a human and a mechanical structure (e.g., see Patent Literature 1).

The observation system in Patent Literature 1 includes an endoscope, a control device, and a display device. The endoscope applies illumination light into a subject, and captures a subject image of the inside of the subject with an imaging element. The control device processes a pixel signal obtained by the imaging of the endoscope, and generates a captured image. The display device displays the captured image obtained by the processing of the control device.

### CITATION LIST

### Patent Literature

Patent Literature 1: JP 2015-142696 A

### SUMMARY

### Technical Problem

Meanwhile, in recent years, a surgical method called hybrid video assisted thoracic surgery (VATS) has been performed. The hybrid VATS is a surgical method performed for, for example, lung cancer, esophagus cancer, and cancer highly infiltrating surrounding organs. In the hybrid VATS, a surgical field can be checked by using a captured image (endoscopic image) displayed on a display device. In addition, the surgical field can be directly viewed with naked eyes with a wide angle as in open-heart surgery.

Unfortunately, when the hybrid VATS is performed by using the observation system in Patent Literature 1, the color of an endoscopic image (color tone of endoscopic image) obtained by imaging a surgical field and displayed on the display device is different from the color in a case where the surgical field is directly viewed with naked eyes (color tone of illumination light applied to surgical field) since the spectral sensitivity of an imaging element is different from that of an eye.

Therefore, there is a demand for a technique capable of improving convenience by bringing the color of an endoscopic image (color tone of endoscopic image) obtained by imaging a surgical field and displayed on the display device and the color in a case where the surgical field is directly viewed with naked eyes (color tone of illumination light applied to surgical field) close to each other.

The present disclosure has been made in view of the above, and an object thereof is to provide an observation system and a control device capable of improving convenience.

### Solution to Problem

To solve the above-described problem and achieve the object, an observation system according to the present disclosure includes: a plurality of light sources configured to apply beams of light in different wavelength bands to a subject; an imaging device configured to image the subject to which illumination light including the beams of light from the plurality of light sources is applied; and a control device configured to control operation of at least one of the plurality of light sources, and generate an output image output to a display device by performing image processing on a pixel signal acquired from the imaging device, wherein the control device includes: a light source control unit configured to control operation of at least one of the plurality of light sources, and adjust a color tone of the illumination light to a first color tone; and an image processing unit configured to perform image processing on the pixel signal, and adjust a color tone of the output image to a second color tone such that the color tone of the output image approaches the first color tone.

A control device according to the present disclosure controls operation of at least one of a plurality of light sources configured to apply beams of light in different wavelength bands to a subject, and generate an output image output to a display device by performing image processing on a pixel signal acquired from an imaging device configured to image the subject to which illumination light including the beams of light from the plurality of light sources is applied, and includes: a light source control unit configured to control the operation of at least one of the plurality of light sources, and adjust a color tone of the illumination light to a first color tone; and an image processing unit configured to perform image processing on the pixel signal, and adjust a color tone of the output image to a second color tone such that the color tone of the output image approaches the first color tone.

### Advantageous Effects of Invention

The observation system and the control device according to the present disclosure can improve convenience.

### BRIEF DESCRIPTION OF DRAWINGS

FIG. 1 illustrates a schematic configuration of an observation system according to a first embodiment.

FIG. 2 is a block diagram illustrating the configuration of a main part of the observation system.

FIG. 3 illustrates the wavelength characteristics of illumination light emitted from a light source device.

FIG. 4 is a flowchart illustrating the operation of the control device.

FIG. 5 is a flowchart illustrating Step S1.

FIG. 6 is a flowchart illustrating Step S2.

FIG. 7 illustrates Variation 1 of the first embodiment.

FIG. 8 is a block diagram illustrating the configuration of the light source device according to a second embodiment.

FIG. 9 illustrates the wavelength characteristics of white light emitted from a fifth visible-light source.

FIG. 10 is a flowchart illustrating Step S1.

FIG. 11 is a flowchart illustrating Step S2.

FIG. 12 illustrates Variation 2 of the second embodiment.

FIG. 13 is a block diagram illustrating the configuration of a main part of the observation system according to the third embodiment.

FIG. 14 illustrates the reflection characteristics of a multiplexer.

FIG. 15 illustrates the wavelength characteristics of illumination light emitted from the light source device.

FIG. 16 is a flowchart illustrating Step S1.

FIG. 17 is a flowchart illustrating Step S2.

FIG. 18 illustrates Variation 3 of the third embodiment.

FIG. 19 illustrates Variation 4 of the first to third embodiments.

FIG. 20 illustrates Variation 5 of the first to third embodiments.

### Description of Embodiments

Embodiments for carrying out the present disclosure (hereinafter, embodiments) will be described below with reference to the drawings. Note that the present disclosure is not limited by the embodiments to be described below. Moreover, in the drawings, the same reference signs are attached to the same parts.

### First Embodiment

### Schematic Configuration of Observation System

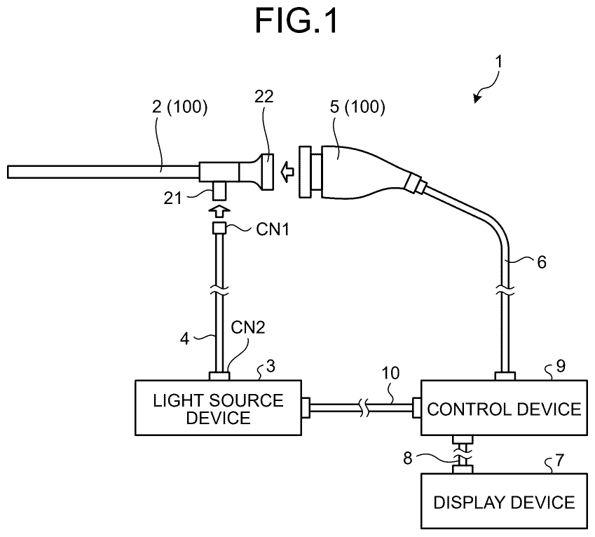

FIG. 1 illustrates a schematic configuration of an observation system 1 according to a first embodiment.

The observation system 1 observes the inside of a living body for medical purposes. As illustrated in FIG. 1, the observation system 1 includes an insertion portion 2, a light source device 3, a light guide 4, a camera head 5, a first transmission cable 6, a display device 7, a second transmission cable 8, a control device 9, and a third transmission cable 10.

The insertion portion 2 corresponds to an endoscope according to the present disclosure. In the first embodiment, the insertion portion 2 includes a rigid endoscope. That is, the insertion portion 2 has an elongated shape. The insertion portion 2 is entirely hard, or has a soft part and the other hard part. The insertion portion 2 is inserted into a living body.

An optical system (not illustrated) is provided in the insertion portion 2. The optical system includes one or a plurality of lenses, and collects a subject image.

Furthermore, as illustrated in FIG. 1, a fiber connection portion 21 is provided on the insertion portion 2. A connector CN1 of the light guide 4 is detachably connected to the fiber connection portion 21.

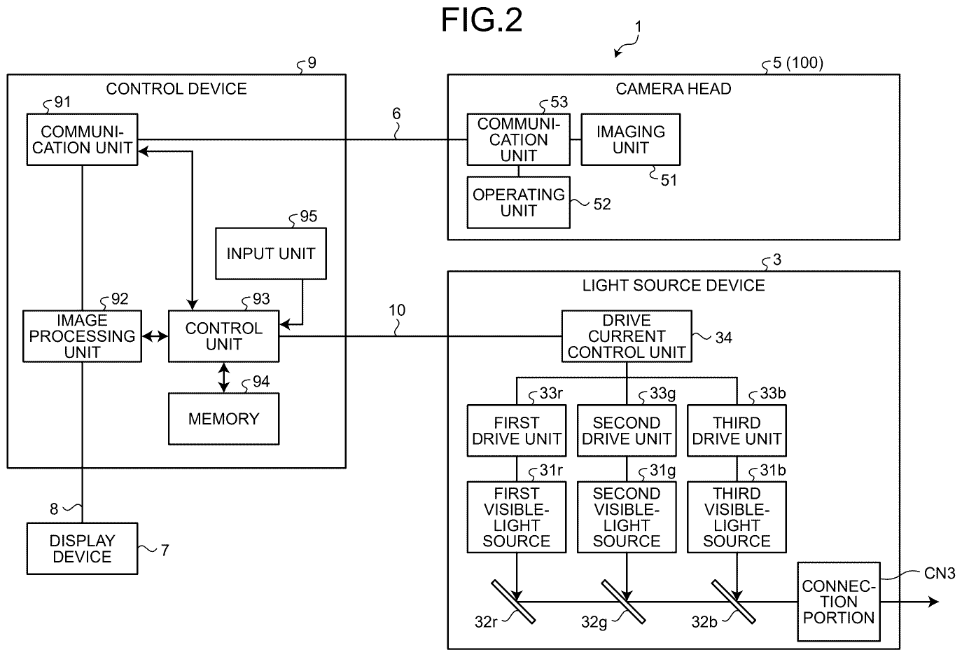

A connection portion CN3 (see FIG. 2) is provided on the light source device 3. A connector CN2 (FIG. 1) of the light guide 4 is detachably connected to the connection portion CN3. The light source device 3 supplies illumination light designated by the control device 9 to an incident end of the light guide 4 under the control of the control device 9. Although the light source device 3 is configured separately from the control device 9 in the first embodiment, this is not a limitation. A configuration in which the light source device 3 is provided inside the control device 9 may be adopted.

Note that the detailed configuration of the light source device 3 will be described in “Configuration of Light Source Device” to be described later.

In the light guide 4, the connector CN1 on the side of the incident end is detachably connected to the fiber connection portion 21, and the connector CN2 on the side of an emission end is detachably connected to the connection portion CN3 of the light source device 3. Then, the light guide 4 supplies the illumination light supplied from the light source device 3 to the insertion portion 2. The illumination light supplied to the insertion portion 2 is applied from a distal end of the insertion portion 2 into the living body. The illumination light (subject image) reflected in the living body is collected by the optical system in the insertion portion 2.

The camera head 5 is detachably connected to an eyepiece portion 22 of the insertion portion 2. The camera head 5 includes an imaging unit 51 (see FIG. 2), an operating unit 52 (see FIG. 2), and a communication unit 53 (see FIG. 2).

The imaging unit 51 includes an imaging element that receives the subject image collected by the insertion portion 2 and that converts the subject image into an electric signal. The imaging element includes a charge coupled device (CCD) and a complementary metal oxide semiconductor (CMOS). Then, the imaging unit 51 outputs a pixel signal (e.g., RAW signal and image signal) obtained by imaging under the control of the control device 9.

The operating unit 52 includes a button and a switch, and receives a user operation from a user such as a surgeon. Then, the operating unit 52 outputs an operation signal in accordance with the user operation.

The communication unit 53 is an interface that communicates with the control device 9 via the first transmission cable 6. The communication unit 53 transmits a pixel signal from the imaging unit 51 and an operation signal from the operating unit 52, and receives a control signal and the like from the control device 9.

One end of the first transmission cable 6 is detachably connected to the control device 9, and the other end thereof is detachably connected to the camera head 5. Then, the first transmission cable 6 transmits a pixel signal and an operation signal transmitted from the communication unit 53 to the control device 9, and transmits, for example, a control signal, a synchronization signal, a clock, and electric power, which are transmitted from the control device 9, to the camera head 5.

Note that, for example, a pixel signal may be transmitted from the camera head 5 to the control device 9 via the first transmission cable 6 by being converted into an optical signal or an electric signal. A control signal, a synchronization signal, and a clock may also be transmitted similarly from the control device 9 to the camera head 5 via the first transmission cable 6.

The display device 7 includes a display formed of, for example, liquid crystal or organic electro luminescence (EL). The display device 7 displays an image based on a video signal from the control device 9 under the control of the control device 9.

One end of the second transmission cable 8 is detachably connected to the display device 7, and the other end thereof is detachably connected to the control device 9. Then, the second transmission cable 8 transmits the video signal that has been processed by the control device 9 to the display device 7.

The control device 9 includes a controller such as a central processing unit (CPU) and a micro processing unit (MPU), and integrally controls operations of the light source device 3, the camera head 5, and the display device 7. Note that the control device 9 may include not only the CPU and the MPU but an integrated circuit such as an application specific integrated circuit (ASIC) and a field-programmable gate array (FPGA).

Note that the detailed configuration of the control device 9 will be described in “Configuration of Control Device” to be described later.

One end of the third transmission cable 10 is detachably connected to the light source device 3, and the other end thereof is detachably connected to the control device 9. Then, the third transmission cable 10 transmits a control signal from the control device 9 to the light source device 3.

### Configuration of Light Source Device

FIG. 2 is a block diagram illustrating the configuration of a main part of the observation system 1.

Next, the configuration of the light source device 3 will be described with reference to FIG. 2.

As illustrated in FIG. 2, the light source device 3 includes first to third visible-light sources 31r, 31g, and 31b, first to third dichroic mirrors 32r, 32g, and 32b, first to third drive units 33r, 33g, and 33b, and a drive current control unit 34.

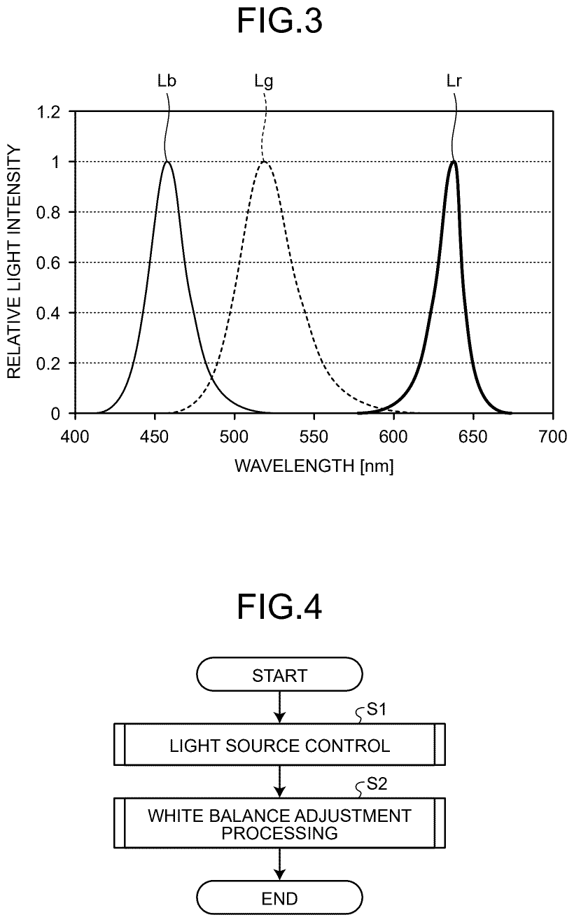

FIG. 3 illustrates the wavelength characteristics of illumination light emitted from the light source device 3. Here, in FIG. 3, the horizontal axis represents a wavelength (nm), and the vertical axis represents relative light intensity. Furthermore, in FIG. 3, a curve Lr indicates the wavelength characteristics of light emitted from the first visible-light source 31r. A curve Lg indicates the wavelength characteristics of light emitted from the second visible-light source 31g. A curve Lb indicates the wavelength characteristics of light emitted from the third visible-light source 31b.

The first to third visible-light sources 31r, 31g, and 31b correspond to a plurality of light sources according to the present disclosure. Then, the first to third visible-light sources 31r, 31g, and 31b emit beams of light in different wavelength bands. The beams of light constitute the illumination light. In the first embodiment, the illumination light is white light.

Specifically, the first visible-light source 31r includes a light emitting diode (LED) that emits light in a red wavelength band (center wavelength: approximately 638 nm) (hereinafter, referred to as red light) as illustrated by the curve Lr in FIG. 3, and emits the red light in a light amount in accordance with supplied drive current.

Furthermore, the second visible-light source 31g includes an LED that emits light in a green wavelength band (center wavelength: approximately 532 nm) (hereinafter, referred to as green light) as illustrated by the curve Lg in FIG. 3, and emits the green light in a light amount in accordance with supplied drive current.

Moreover, the third visible-light source 31b includes an LED that emits light in a blue wavelength band (center wavelength: approximately 465 nm) (hereinafter, referred to as blue light) as illustrated by the curve Lb in FIG. 3, and emits the blue light in a light amount in accordance with supplied drive current.

Note that each of the first to third visible-light sources 31r, 31g, and 31b may include one LED or a plurality of LEDs. Furthermore, the first to third visible-light sources 31r, 31g, and 31b may include not only LEDs but semiconductor lasers.

The first dichroic mirror 32r reflects red light, and transmits light in a wavelength band different from that of the red light. Note that a mirror that reflects light in substantially all wavelength bands may be adopted instead of the first dichroic mirror 32r.

The second dichroic mirror 32g transmits red light reflected by the first dichroic mirror 32r, and reflects green light in the same direction as the traveling direction of the red light.

The third dichroic mirror 32b transmits the red light transmitted through the second dichroic mirror 32g and the green light reflected by the second dichroic mirror 32g, and reflects blue light in the same direction as the traveling directions of the red light and the green light.

Then, the red light, the green light, and the blue light, whose traveling directions are aligned by the first to third dichroic mirrors 32r, 32g, and 32b, are let in to the incident end of the light guide 4 connected to the connection portion CN3.

The first drive unit 33r supplies drive current designated by the drive current control unit 34 to the first visible-light source 31r to drive the first visible-light source 31r.

The second drive unit 33g supplies drive current designated by the drive current control unit 34 to the second visible-light source 31g to drive the second visible-light source 31g.

The third drive unit 33b supplies drive current designated by the drive current control unit 34 to the third visible-light source 31b to drive the third visible-light source 31b.

The drive current control unit 34 causes drive currents to be supplied from the first to third drive units 33r, 33g, and 33b to the first to third visible-light sources 31r, 31g, and 31b under the control of the control device 9.

The insertion portion 2 and the camera head 5 described above correspond to an imaging device 100 (FIGS. 1 and 2) according to the present disclosure.

### Configuration of Control Device

Next, the configuration of the control device 9 will be described with reference to FIG. 2.

As illustrated in FIG. 2, the control device 9 includes a communication unit 91, an image processing unit 92, a control unit 93, a memory 94, and an input unit 95.

The communication unit 91 is an interface that communicates with the camera head 5 (communication unit 53) via the first transmission cable 6. The communication unit 91 receives a pixel signal from the imaging unit 51 and an operation signal from the operating unit 52, and transmits a control signal and the like.

The image processing unit 92 generates a captured image based on a pixel signal received by the communication unit 91 by executing image processing on the pixel signal. The image processing unit 92 generates a display video signal for displaying the captured image on the display device 7. The image processing unit 92 outputs the video signal to the display device 7 via the second transmission cable 8. The captured image corresponds to an output image according to the present disclosure.

Here, examples of the image processing can include digital gain processing, optical black subtraction processing, white balance (WB) adjustment processing, demosaic processing, color correction matrix processing, gamma correction processing, YC conversion processing, and enlargement processing (electronic zoom). In the digital gain processing, a pixel signal (digital signal) received by the communication unit 91 is multiplied by a digital gain that amplifies the digital signal. In the YC conversion processing, a luminance signal and a color difference signal (Y, Cb/Cr signal) are generated.

Note that the white balance adjustment processing is image processing of adjusting the color tone of a captured image by performing image processing on a pixel signal. Details thereof will be described in “Operation of Control Device” to be described later.

Furthermore, the image processing unit 92 executes detection processing to be described below.

In the detection processing, detection of the contrast and frequency component of an image within a detection region, detection of a luminance average value and the maximum and minimum pixels within the detection region with a filter and the like, determination through comparison with a threshold, and detection of a histogram and the like are executed based on pixel information (e.g., Y value (luminance signal (Y signal))) on each pixel within a specific detection region of the entire image region of a captured image after the above-described image processing is executed. For example, the detection region is a rectangular region centered on the image center of the captured image. Then, the image processing unit 92 outputs detection information (e.g., contrast, frequency component, luminance average value, maximum and minimum pixels, and histogram) obtained by the detection processing to the control unit 93.

The control unit 93 is implemented by a controller, such as a CPU and an MPU, executing various programs stored in the memory 94. The control unit 93 controls the operations of the light source device 3, the camera head 5, and the display device 7, and controls the operation of the entire control device 9. Note that the control unit 93 may include not only a CPU and an MPU but an integrated circuit such as an ASIC and an FPGA. The control unit 93 has a function of a light source control unit according to the present disclosure. The light source control unit controls the operation of at least one of the first to third visible-light sources 31r, 31g, and 31b.

Note that the function of the light source control unit will be described in “Operation of Control Device” to be described later.

The memory 94 stores a program to be executed by the control unit 93, information necessary for processing of the control unit 93, and the like. Examples of the information necessary for processing of the control unit 93 can include first and second pieces of color tone correction information.

Note that details of the first and second pieces of color tone correction information will be described in “Operation of Control Device” to be described later.

The input unit 95 includes an operation device such as a mouse, a keyboard, and a touch panel, and receives a user operation from a user such as a surgeon. Then, the input unit 95 outputs an operation signal in accordance with the user operation to the control unit 93.

### Operation of Control Device

Next, operations of the above-described control device 9 will be described.

Note that, for convenience of description, only control for adjusting the color tone of illumination light (hereinafter, referred to as light source control) and the white balance adjustment processing for adjusting the color tone of a captured image will be described below as the operations of the control device 9.

FIG. 4 is a flowchart illustrating an operation of the control device 9.

First, the control unit 93 (light source control unit) executes light source control with reference to the first color tone correction information stored in the memory 94 (Step S1).

Here, the first color tone correction information relates to light amounts (corresponding to drive currents to be supplied to first to third visible-light sources 31r, 31g, and 31b) in accordance with a light amount ratio of red light, green light, and blue light from the first to third visible-light sources 31r, 31g, and 31b. The light amount ratio is set based on the spectral sensitivity of a human eye. When red light, green light, and blue light in the light amounts in accordance with the light amount ratio are applied to the inside of a living body, the inside of the living body has a color suitable for the user such as a surgeon to view an affected area such as a tumor without a feeling of strangeness in a case where the user directly views the inside of the living body with naked eyes. That is, when the first to third visible-light sources 31r, 31g, and 31b emit red light, green light, and blue light in light amounts in accordance with the light amount ratio, the color tone of illumination light to be applied into the living body is adjusted to a desired color tone (first color tone).

Note that the first color tone correction information is provided for each luminance of illumination light to be adjusted in dimming control included in light source control executed by the control unit 93. Here, a light amount ratio of red light, green light, and blue light in the first color tone correction information corresponding to the luminance of one beam of illumination light is different from a light amount ratio of red light, green light, and blue light in the first color tone correction information corresponding to the luminance of another beam of illumination light.

Step S1 will be specifically described below.

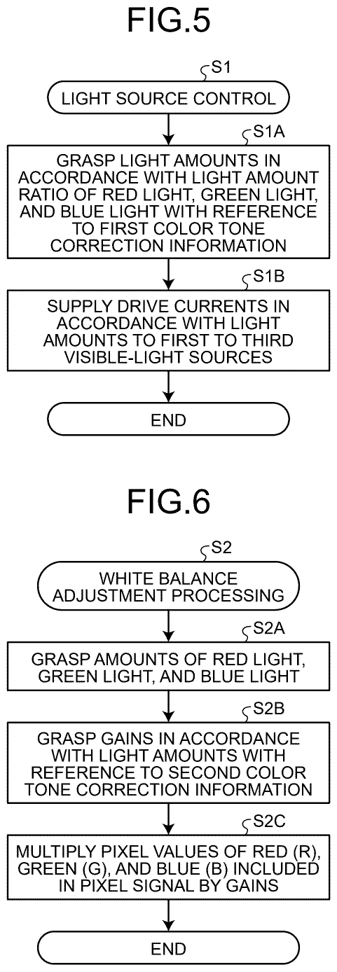

FIG. 5 is a flowchart illustrating Step S1.

The control unit 93 grasps the light amounts (corresponding to drive currents to be supplied to first to third visible-light sources 31r, 31g, and 31b) in accordance with a light amount ratio of red light, green light, and blue light with reference to the first color tone correction information stored in the memory 94 (Step S1A).

After Step S1A, the control unit 93 controls the operation of the light source device 3, and supplies drive currents in accordance with the light amounts to the first to third visible-light sources 31r, 31g, and 31b (Step S1B). This causes red light, green light, and blue light in the light amounts to be emitted from the first to third visible-light sources 31r, 31g, and 31b.

Here, the above-described dimming control is as follows.

The control unit 93 calculates a brightness evaluation value for changing the brightness of an image within a detection region of the entire image region of a captured image to a reference brightness (changing detection information to reference luminance average value) based on detection information output from the image processing unit 92. The brightness evaluation value corresponds to the luminance of illumination light. Then, the control unit 93 executes dimming control of adjusting the luminance of illumination light so as to adjust the brightness of the image within the detection region of the entire image region of the captured image to the reference brightness.

When executing the above-described dimming control, the control unit 93 refers to the first color tone correction information corresponding to the luminance of illumination light to be adjusted under the dimming control in Step S1.

After Step S1, the image processing unit 92 executes white balance adjustment processing with reference to the second color tone correction information stored in the memory 94 (Step S2).

Here, the second color tone correction information includes a lookup table generated in consideration of the spectral sensitivity of the imaging unit 51 and the display characteristics of the display device 7. In the second color tone correction information, light amounts (light amount ratio) of red light, green light, and blue light emitted from the first to third visible-light sources 31r, 31g, and 31b are associated with gains. Examples of the display characteristics of the display device 7 can include the color balance and a gamma curve of the display device 7. Furthermore, pixel values of red (R), green (G), and blue (B) included in a pixel signal are multiplied by the gains in the white balance adjustment processing.

Note that the spectral sensitivity of the imaging unit 51 corresponds to the imaging characteristics of the imaging device (spectral sensitivity of imaging device) according to the present disclosure. Furthermore, the light amounts of red light, green light, and blue light respectively emitted from the first to third visible-light sources 31r, 31g, and 31b correspond to the characteristics of a light source according to the present disclosure. Then, the above-described second color tone correction information is not required to be generated in consideration of the display characteristics of the display device 7 as long as the second color tone correction information includes a lookup table generated in consideration of the imaging characteristics of the imaging device according to the present disclosure. Furthermore, in the above-described second color tone correction information, information associated with the gains is not limited to the light amounts of red light, green light, and blue light respectively emitted from the first to third visible-light sources 31r, 31g, and 31b. Drive currents to be supplied to the first to third visible-light sources 31r, 31g, and 31b or the color tone of illumination light may be adopted as long as the drive currents or the color tone of the illumination light corresponds to the characteristics of the light source according to the present disclosure. Moreover, in the white balance adjustment processing, at least one of pixel values of the red (R), the green (G), and the blue (B) included in a pixel signal is required to be multiplied by a gain. All the pixel values of the red (R), the green (G), and the blue (B) are not required to be multiplied by the gain.

Step S2 will be specifically described below.

FIG. 6 is a flowchart illustrating Step S2.

The image processing unit 92 grasps the amounts of red light, green light, and blue light, which have been caused to be emitted from the first to third visible-light sources 31r, 31g, and 31b by the control unit 93 in Step S1 (Step S2A).

After Step S2A, the image processing unit 92 grasps gains in accordance with the light amounts grasped in Step S2A with reference to the second color tone correction information stored in the memory 94 (Step S2B).

After Step S2B, the image processing unit 92 executes white balance adjustment processing of multiplying the pixel values of the red (R), the green (G), and the blue (B) included in the pixel signal by the gains grasped in Step S2B (Step S2C). This causes the color tone of the captured image to be adjusted to a desired color tone (second color tone).

Then, the first color tone and the second color tone are substantially the same. That is, in Step S1, the control unit 93 controls the operation of the light source device 3, and adjusts the color tone of the illumination light to the first color tone. In contrast, in Step S2, the image processing unit 92 performs white balance adjustment processing on the pixel signal, and adjusts the color tone of the captured image to the second color tone approaching the first color tone.

According to the above-described first embodiment, the following effects are exhibited.

In the observation system 1 according to the first embodiment, the control unit 93 controls the operations of the first to third visible-light sources 31r, 31g, and 31b, and adjusts the color tone of the illumination light to the first color tone. Furthermore, the image processing unit 92 performs image processing on the pixel signal, and adjusts the color tone of the captured image to the second color tone approaching the first color tone.

Therefore, according to the observation system 1 of the first embodiment, in the case of performing hybrid VATS, the color of a captured image (color tone of captured image (second color tone)) can be adjusted to a color (color tone) close to a color in the case where a surgical field is directly viewed with naked eyes (color tone of illumination light applied to surgical field (first color tone)). The captured image contains the surgical field, and is displayed on the display device 7. Convenience can thus be improved.

In particular, the control unit 93 adjusts the color tone of the illumination light to the first color tone by controlling the light source device 3 using the first color tone correction information in consideration of the spectral sensitivity of an eye. Furthermore, the image processing unit 92 adjusts the color tone of the captured image to the second color tone by performing image processing on the pixel signal using the second color tone correction information in consideration of the spectral sensitivity of the imaging unit 51 and the display characteristics of the display device 7.

Therefore, the color tone of the illumination light and the color tone of the captured image can be adjusted to a color tone suitable for the user such as a surgeon to view an affected area such as a tumor without a feeling of strangeness.

### Variation 1

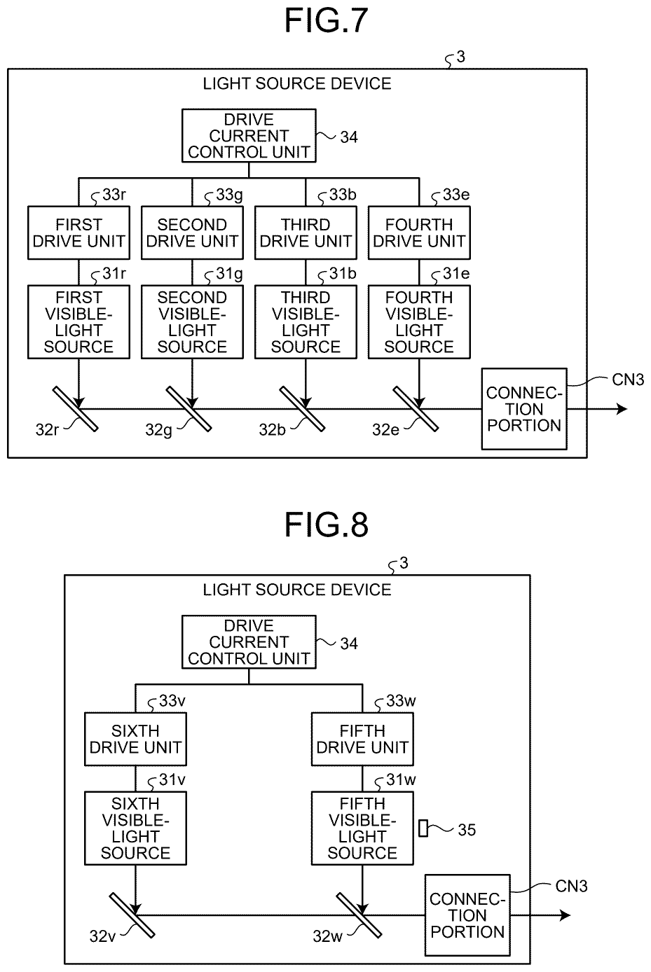

FIG. 7 illustrates Variation 1 of the first embodiment. Specifically, FIG. 7 is a block diagram illustrating the configuration of the light source device 3 according to Variation 1.

In the above-described first embodiment, the first to third visible-light sources 31r, 31g, and 31b are adopted as a plurality of light sources according to the present disclosure, this is not a limitation. The configuration of the light source device 3 according to Variation 1 in FIG. 7 may be adopted.

As illustrated in FIG. 7, the light source device 3 according to Variation 1 includes a fourth visible-light source 31e, a fourth dichroic mirror 32e, and a fourth drive unit 33e in addition to the first to third visible-light sources 31r, 31g, and 31b, the first to third dichroic mirrors 32r, 32g, and 32b, the first to third drive units 33r, 33g, and 33b, and the drive current control unit 34, which have been described above in the first embodiment.

The fourth visible-light source 31e emits light (hereinafter, referred to as additional light) in a wavelength band, which is insufficient for making white illumination light, in consideration of the wavelength characteristics of red light, green light, and blue light respectively emitted from the first to third visible-light sources 31r, 31g, and 31b. For example, the fourth visible-light source 31e includes an LED or a semiconductor laser that emits additional light of a center wavelength: approximately 405 nm on the side of a shorter wavelength than the center wavelength of blue light or a center wavelength: approximately 680 nm on the side of a longer wavelength than the center wavelength of red light. The fourth visible-light source 31e emits the additional light in a light amount in accordance with supplied drive current.

Note that the fourth visible-light source 31e may include one LED or one semiconductor laser, or a plurality of LEDs or a plurality of semiconductor lasers.

The fourth dichroic mirror 32e transmits red light and green light transmitted through the third dichroic mirror 32b and blue light reflected by the third dichroic mirror 32b, and reflects additional light in the same direction as the traveling directions of the red light, the green light, and the blue light.

Then, the red light, the green light, the blue light, and the additional light, whose traveling directions are aligned by the first to fourth dichroic mirrors 32r, 32g, 32b, and 32e, are let in to the incident end of the light guide 4 connected to the connection portion CN3.

The fourth drive unit 33e supplies drive current designated by the drive current control unit 34 to the fourth visible-light source 31e to drive the fourth visible-light source 31e.

The drive current control unit 34 according to Variation 1 causes drive currents to be supplied from the first to fourth drive units 33r, 33g, 33b, and 33e to the first to fourth visible-light sources 31r, 31g, 31b, and 31e under the control of the control unit 93.

In Step S1 according to Variation 1, the control unit 93 executes the following first light source control or second light source control.

Here, the first color tone correction information to be used when the first light source control is executed relates to light amounts (corresponding to drive currents to be supplied to first to fourth visible-light sources 31r, 31g, 31b, and 31e) in accordance with a light amount ratio of red light, green light, blue light, and additional light from the first to fourth visible-light sources 31r, 31g, 31b, and 31e. The light amount ratio is set based on the spectral sensitivity of a human eye. When red light, green light, blue light, and additional light in the light amounts in accordance with the light amount ratio are applied to the inside of a living body, the inside of the living body has a color suitable for the user such as a surgeon to view an affected area such as a tumor without a feeling of strangeness in a case where the user directly views the inside of the living body with naked eyes. That is, when the first to fourth visible-light sources 31r, 31g, 31b, and 31e emit red light, green light, blue light, and additional light in light amounts in accordance with the light amount ratio, the color tone of illumination light to be applied into the living body is adjusted to the first color tone.

Note that the first color tone correction information to be used when the first light source control is executed is provided for each luminance of illumination light to be adjusted in dimming control executed by the control unit 93. Here, a light amount ratio of red light, green light, and blue light in the first color tone correction information corresponding to the luminance of one beam of illumination light is the same as a light amount ratio of red light, green light, and blue light in the first color tone correction information corresponding to the luminance of another beam of illumination light. That is, a light amount ratio of red light, green light, and blue light is fixed. In contrast, a light amount of additional light in the first color tone correction information corresponding to the luminance of one beam of illumination light is different from a light amount of additional light in the first color tone correction information corresponding to the luminance of another beam of illumination light. That is, when the luminance of illumination light is adjusted, the color tone of the illumination light is adjusted to the first color tone by changing the light amount of the additional light.

Then, in Step S1, the control unit 93 executes the first light source control with reference to the first color tone correction information stored in the memory 94.

Specifically, the control unit 93 grasps the light amounts (corresponding to drive currents to be supplied to first to fourth visible-light sources 31r, 31g, 31b, and 31e) in accordance with a light amount ratio of red light, green light, blue light, and additional light with reference to the first color tone correction information stored in the memory 94. Then, the control unit 93 controls the operation of the light source device 3 to supply drive currents in accordance with the light amounts to the first to fourth visible-light sources 31r, 31g, 31b, and 31e. The control unit 93 causes red light, green light, blue light, and additional light in the light amounts to be emitted from the first to fourth visible-light sources 31r, 31g, 31b, and 31e.

Note that, when executing the dimming control, the control unit 93 refers to the first color tone correction information corresponding to the luminance of illumination light to be adjusted under the dimming control in Step S1.

Furthermore, the first color tone correction information to be used when the second light source control is executed relates to light amounts in accordance with a light amount ratio of red light, green light, blue light, and additional light from the first to fourth visible-light sources 31r, 31g, 31b, and 31e. The light amount ratio is set based on the spectral sensitivity of a human eye. When red light, green light, blue light, and additional light in the light amounts in accordance with the light amount ratio are applied to the inside of a living body, the inside of the living body has a color suitable for the user such as a surgeon to view an affected area such as a tumor without a feeling of strangeness in a case where the user directly views the inside of the living body with naked eyes. That is, when the first to fourth visible-light sources 31r, 31g, 31b, and 31e emit red light, green light, blue light, and additional light in light amounts in accordance with the light amount ratio, the color tone of illumination light to be applied into the living body is adjusted to the first color tone.

Note that the first color tone correction information to be used when the second light source control is executed is provided for each luminance of illumination light to be adjusted in dimming control executed by the control unit 93. Here, a light amount ratio of red light, green light, blue light, and additional light in the first color tone correction information corresponding to the luminance of one beam of illumination light is different from a light amount ratio of red light, green light, blue light, and additional light in the first color tone correction information corresponding to the luminance of another beam of illumination light. That is, when the luminance of illumination light is adjusted, the color tone of the illumination light is adjusted to the first color tone by changing the light amount ratio of red light, green light, blue light, and additional light.

Then, in Step S1, the control unit 93 executes the second light source control with reference to the first color tone correction information stored in the memory 94.

Specifically, the control unit 93 grasps the light amounts in accordance with a light amount ratio of red light, green light, blue light, and additional light with reference to the first color tone correction information stored in the memory 94. Then, the control unit 93 controls the operation of the light source device 3 to supply drive currents in accordance with the light amounts to the first to fourth visible-light sources 31r, 31g, 31b, and 31e. The control unit 93 causes red light, green light, blue light, and additional light in the light amounts to be emitted from the first to fourth visible-light sources 31r, 31g, 31b, and 31e.

Note that, when executing the dimming control, the control unit 93 refers to the first color tone correction information corresponding to the luminance of illumination light to be adjusted under the dimming control in Step S1.

Furthermore, in Step S2 according to Variation 1, the image processing unit 92 executes the following white balance adjustment processing.

Here, the second color tone correction information to be used in the white balance adjustment processing includes a lookup table generated in consideration of the spectral sensitivity of the imaging unit 51 and the display characteristics of the display device 7. In the second color tone correction information, light amounts (light amount ratio) of red light, green light, blue light, and additional light emitted from the first to fourth visible-light sources 31r, 31g, 31b, and 31e are associated with gains. Examples of the display characteristics of the display device 7 can include the color balance and a gamma curve of the display device 7. Furthermore, pixel values of red (R), green (G), and blue (B) included in a pixel signal are multiplied by the gains in the white balance adjustment processing.

Note that, the light amounts of red light, green light, blue light, and additional light respectively emitted from the first to fourth visible-light sources 31r, 31g, 31b, and 31e correspond to the characteristics of a light source according to the present disclosure. Then, the above-described second color tone correction information is not required to be generated in consideration of the display characteristics of the display device 7 as long as the second color tone correction information includes a lookup table generated in consideration of the imaging characteristics of the imaging device according to the present disclosure. Furthermore, in the above-described second color tone correction information, information associated with the gains is not limited to the light amounts of red light, green light, blue light, and additional light respectively emitted from the first to fourth visible-light sources 31r, 31g, 31b, and 31e. Drive currents to be supplied to the first to fourth visible-light sources 31r, 31g, 31b, and 31e or the color tone of illumination light may be adopted as long as the drive currents the color tone of the illumination light corresponds to the characteristics of the light source according to the present disclosure.

Then, in Step S2, the image processing unit 92 grasps the amounts of red light, green light, blue light, and additional light, which have been caused to be emitted from the first to fourth visible-light sources 31r, 31g, 31b, and 31e by the control unit 93 in Step S1. Furthermore, the image processing unit 92 grasps gains of the grasped light amounts with reference to the second color tone correction information stored in the memory 94. Then, the image processing unit 92 executes white balance adjustment processing of multiplying the pixel values of the red (R), the green (G), and the blue (B) included in the pixel signal by the grasped gains. This causes the color tone of the captured image to be adjusted to the second color tone.

Even when the configuration of Variation 1 described above is adopted, effects similar to those of the above-described first embodiment are exhibited.

### Second Embodiment

Next, a second embodiment will be described.

In the following description, the same reference signs are attached to configurations similar to those in the above-described first embodiment, and detailed description thereof will be omitted or simplified.

FIG. 8 is a block diagram illustrating the configuration of the light source device 3 according to the second embodiment.

As illustrated in FIG. 8, the second embodiment is different from the above-described first embodiment in the configuration of the light source device 3.

As illustrated in FIG. 8, the light source device 3 according to the second embodiment includes a fifth visible-light source 31w, a sixth visible-light source 31v, fifth and sixth dichroic mirrors 32w and 32v, fifth and sixth drive units 33w and 33v, a drive current control unit 34, and a temperature measurement unit 35.

The fifth and sixth visible-light sources 31w and 31v correspond to a plurality of light sources according to the present disclosure. Then, the fifth and sixth visible-light sources 31w and 31v emit beams of light in different wavelength bands. The beams of light constitute the illumination light.

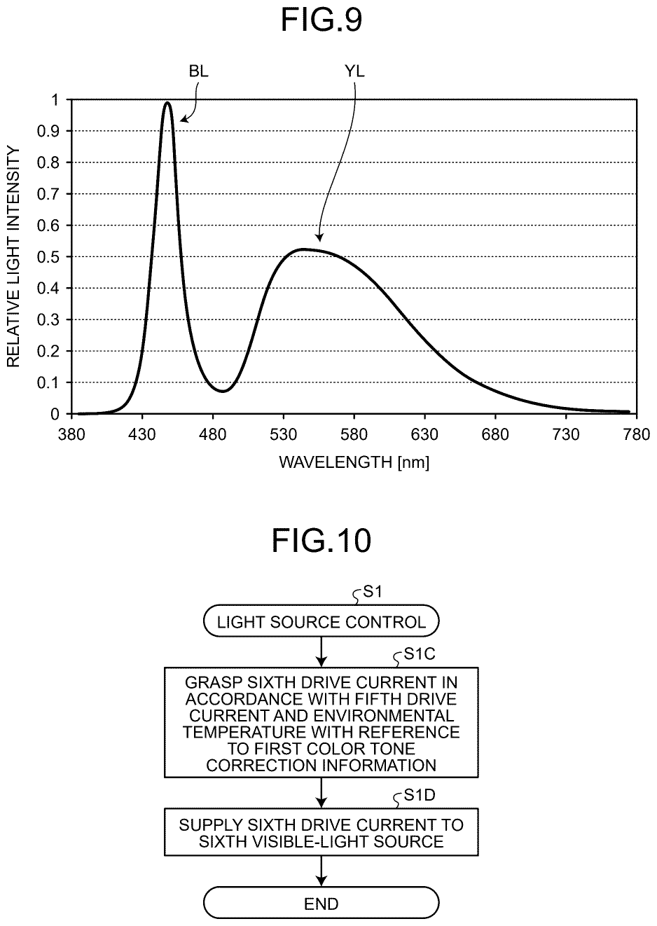

FIG. 9 illustrates the wavelength characteristics of illumination light emitted from the fifth visible-light source 31w. Here, in FIG. 9, the horizontal axis represents a wavelength (nm), and the vertical axis represents relative light intensity.

Specifically, the fifth visible-light source 31w corresponds to a first light source according to the present disclosure. In the second embodiment, the fifth visible-light source 31w includes an LED or a semiconductor laser that emits white light. The fifth visible-light source 31w emits the white light in a light amount in accordance with supplied drive current. More specifically, the fifth visible-light source 31w has a configuration in which a blue LED or a blue semiconductor laser that emits blue light BL (FIG. 9) in a blue wavelength band is mounted in a package cavity and a phosphor such as YAGCe is enclosed around the blue LED or the blue semiconductor laser. Then, the phosphor is excited by the blue light BL emitted from the blue LED or the blue semiconductor laser. The phosphor emits fluorescent light YL (FIG. 9) in a yellow wavelength band. That is, the fifth visible-light source 31w emits white light in which the blue light BL and the fluorescent light YL are mixed.

Note that the fifth visible-light source 31w may include one LED or one semiconductor laser, or a plurality of LEDs or a plurality of semiconductor lasers.

Here, the fifth visible-light source 31w has the following characteristics.

That is, the relative light intensity of the blue light BL and the relative light intensity of the fluorescent light YL are changed by drive current supplied to the fifth visible-light source 31w. Specifically, as the drive current decreases, the relative light intensity of the blue light BL and the relative light intensity of the fluorescent light YL decrease.

Furthermore, the center wavelength of the blue light BL changes depending on environmental temperature around the fifth visible-light source 31w. Specifically, when the environmental temperature increases, the center wavelength of the blue light BL decreases. In contrast, when the environmental temperature decreases, the center wavelength of the blue light BL increases. Furthermore, the relative light intensity of the fluorescent light YL changes depending on environmental temperature around the fifth visible-light source 31w. Specifically, when the environmental temperature increases, the relative light intensity of the fluorescent light YL decreases. In contrast, when the environmental temperature decreases, the relative light intensity of the fluorescent light YL increases.

Adjusting the color tone of white light (blue light BL and fluorescent light YL) emitted from the fifth visible-light source 31w to the first color tone is difficult due to the above-described characteristics of the fifth visible-light source 31w.

Therefore, the sixth visible-light source 31v is provided to adjust the color tone of illumination light emitted from the light source device 3 to the first color tone. That is, the sixth visible-light source 31v corresponds to a second light source according to the present disclosure. In the second embodiment, the sixth visible-light source 31v includes an LED or a semiconductor laser that emits light (hereinafter, referred to as adjustment light) of a center wavelength: approximately 405 nm on the side of shorter wavelength than the center wavelength of blue light BL. The sixth visible-light source 31v emits the adjustment light in a light amount in accordance with supplied drive current.

Note that the sixth visible-light source 31v may emit not only adjustment light of a center wavelength of approximately 405 nm but adjustment light in another wavelength band as long as the adjustment light has a center wavelength different from the emission spectra of the blue light BL and the fluorescent light YL. For example, the sixth visible-light source 31v may be adopted. The sixth visible-light source 31v emits adjustment light in a wavelength band of 430 nm or less, 450 to 500 nm, or approximately 680 to 700 nm. Furthermore, the sixth visible-light source 31v may include one LED or one semiconductor laser, or a plurality of LEDs or a plurality of semiconductor lasers.

The sixth dichroic mirror 32v reflects adjustment light, and transmits light in a wavelength band different from that of the adjustment light. Note that a mirror that reflects light in substantially all wavelength bands may be adopted instead of the sixth dichroic mirror 32v.

The fifth dichroic mirror 32w transmits adjustment light reflected by the sixth dichroic mirror 32v, and reflects white light (blue light BL and fluorescent light YL) in the same direction as the traveling direction of the adjustment light.

Then, the white light (blue light BL and fluorescent light YL) and adjustment light, whose traveling directions are aligned by the fifth and sixth dichroic mirrors 32w and 32v, are let in to the incident end of the light guide 4 connected to the connection portion CN3.

The fifth drive unit 33w supplies drive current designated by the drive current control unit 34 to the fifth visible-light source 31w to drive the fifth visible-light source 31w.

The sixth drive unit 33v supplies drive current designated by the drive current control unit 34 to the sixth visible-light source 31v to drive the sixth visible-light source 31v.

The drive current control unit 34 according to the second embodiment causes drive currents to be supplied from the fifth and sixth drive units 33w and 33v to the fifth and sixth visible-light sources 31w and 31v under the control of the control unit 93.

The temperature measurement unit 35 is a temperature sensor such as a thermocouple and a thermistor. The temperature measurement unit 35 measures the environmental temperature around the fifth visible-light source 31w under the control of the control unit 93. Then, the temperature measurement unit 35 outputs a signal in accordance with the measured environmental temperature to the control unit 93.

In Step S1 according to the second embodiment, the control unit 93 executes the following light source control.

FIG. 10 is a flowchart illustrating Step S1.

Here, the first color tone correction information to be used when the light source control is executed includes a lookup table generated in consideration of the spectral sensitivity of a human eye. In the first color tone correction information, drive current supplied to the fifth visible-light source 31w (hereinafter, referred to as fifth drive current) and the environmental temperature measured by the temperature measurement unit 35 are associated with the sixth drive current. The sixth drive current is supplied to the sixth visible-light source 31v. When the fifth drive current is supplied to the fifth visible-light source 31w and the fifth visible-light source 31w applies white light into a living body, the sixth drive current corresponding to the fifth drive current in the first color tone correction information is supplied to the sixth visible-light source 31v, and the sixth visible-light source 31v applies adjustment light into the living body. The living body thereby has a color suitable for the user such as a surgeon to view an affected area such as a tumor without a feeling of strangeness in a case where the user directly views the inside of the living body with naked eyes. That is, when the fifth and sixth drive currents are respectively supplied to the fifth and sixth visible-light sources 31w and 31v, the color tone of illumination light to be applied into the living body is adjusted to the first color tone.

Note that the first color tone correction information to be used when the light source control is executed is provided for each luminance of illumination light to be adjusted in dimming control executed by the control unit 93. Here, the sixth drive current in the first color tone correction information corresponding to the luminance of one beam of illumination light is different from the sixth drive current in the first color tone correction information corresponding to the luminance of another beam of illumination light. That is, when the fifth drive current to be supplied to the fifth visible-light source 31w is changed in order to adjust the luminance of illumination light, the sixth drive current is also changed. The color tone of the illumination light is adjusted to the first color tone by changing a light amount of the adjustment light.

Then, in Step S1, the control unit 93 executes the light source control with reference to the first color tone correction information stored in the memory 94.

Specifically, the control unit 93 controls the operation of the light source device 3, and supplies the fifth drive current to the fifth visible-light source 31w. The control unit 93 grasps the sixth drive current in accordance with the fifth drive current and the environmental temperature measured by the temperature measurement unit 35 with reference to the first color tone correction information stored in the memory 94 (Step S1C).

After Step S1C, the control unit 93 controls the operation of the light source device 3, and supplies the sixth drive current grasped in Step S1C to the sixth visible-light source 31v (Step S1D).

Note that, when executing the dimming control, the control unit 93 refers to the first color tone correction information corresponding to the luminance of illumination light to be adjusted under the dimming control in Step S1.

Furthermore, in Step S2 according to the second embodiment, the image processing unit 92 executes the following white balance adjustment processing.

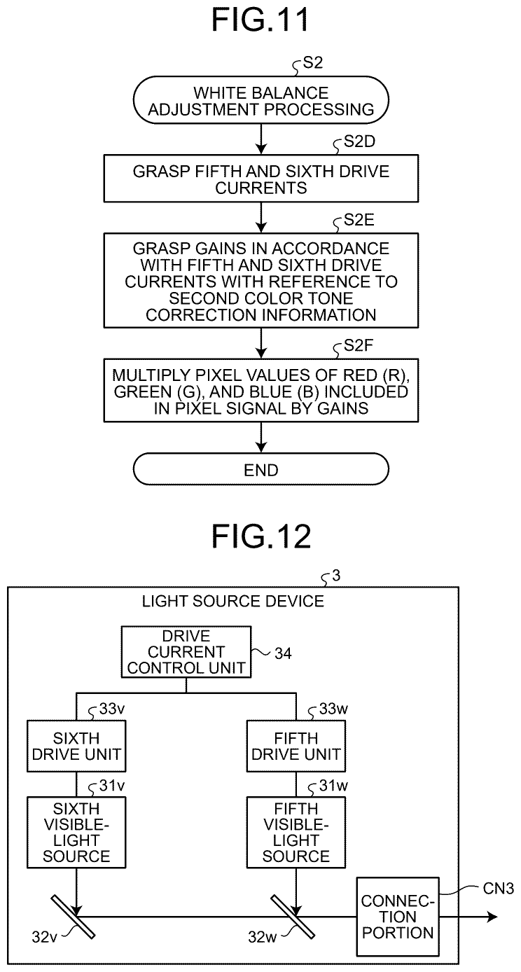

FIG. 11 is a flowchart illustrating Step S2.

Here, the second color tone correction information to be used in the white balance adjustment processing includes a lookup table generated in consideration of the spectral sensitivity of the imaging unit 51 and the display characteristics of the display device 7. In the second color tone correction information, the fifth and sixth drive currents respectively supplied to the fifth and sixth visible-light sources 31w and 31v and gains are associated with each other. Examples of the display characteristics of the display device 7 can include the color balance and a gamma curve of the display device 7. Furthermore, pixel values of red (R), green (G), and blue (B) included in a pixel signal are multiplied by the gains in the white balance adjustment.

Note that the fifth and sixth drive currents respectively supplied to the fifth and sixth visible-light sources 31w and 31v correspond to the characteristics of a light source according to the present disclosure. Then, the above-described second color tone correction information is not required to be generated in consideration of the display characteristics of the display device 7 as long as the second color tone correction information includes a lookup table generated in consideration of the imaging characteristics of the imaging device according to the present disclosure. Furthermore, in the above-described second color tone correction information, information associated with the gains is not limited to the fifth and sixth drive currents respectively supplied to the fifth and sixth visible-light sources 31w and 31v. Light amounts of white light (blue light BL and fluorescent light YL) and adjustment light respectively emitted from the fifth and sixth visible-light sources 31w and 31v or the color tone of illumination light may be adopted as long as the light amounts or the color tone corresponds to the characteristics of the light source according to the present disclosure.

Then, in Step S2, the image processing unit 92 grasps the fifth and sixth drive currents respectively supplied by the control unit 93 to the fifth and sixth visible-light sources 31w and 31v in Step S1 (Step S2D).

After Step S2D, the image processing unit 92 grasps gains in accordance with the grasped fifth and sixth drive currents with reference to the second color tone correction information stored in the memory 94 (Step S2E).

After Step S2E, the image processing unit 92 executes white balance adjustment processing of multiplying the pixel values of the red (R), the green (G), and the blue (B) included in the pixel signal by the grasped gains (Step S2F). This causes the color tone of the captured image to be adjusted to the second color tone.

According to the above-described second embodiment, the following effect is exhibited in addition to effects similar to those in the above-described first embodiment.

In the observation system 1 according to the second embodiment, even when the fifth visible-light source 31w, in which the color tone of white light (blue light BL and fluorescent light YL) changes depending on drive current and environmental temperature, is used, the color tone of illumination light (white light (blue light BL and fluorescent light YL) and adjustment light) can be adjusted to the first color tone by controlling the operation of the sixth visible-light source 31v.

In particular, the first color tone correction information adopts the fifth drive current and environmental temperature as elements for guiding the sixth drive current. Therefore, the color tone of illumination light can be favorably adjusted to the first color tone by controlling the light source device 3 using the first color tone correction information.

### Variation 2

FIG. 12 illustrates Variation 2 of the second embodiment. Specifically, FIG. 12 is a block diagram illustrating the configuration of the light source device 3 according to Variation 2.

Although, in the above-described second embodiment, the first color tone correction information adopts both the fifth drive current and environmental temperature as elements for guiding the sixth drive current, this is not a limitation. Only the fifth drive current may be adopted. That is, in the above-described second embodiment, as in Variation 2 in FIG. 12, the light source device 3 in which the temperature measurement unit 35 is not provided may be adopted.

### Third Embodiment

Next, a third embodiment will be described.

In the following description, the same reference signs are attached to configurations similar to those in the above-described first and second embodiments, and detailed description thereof will be omitted or simplified.

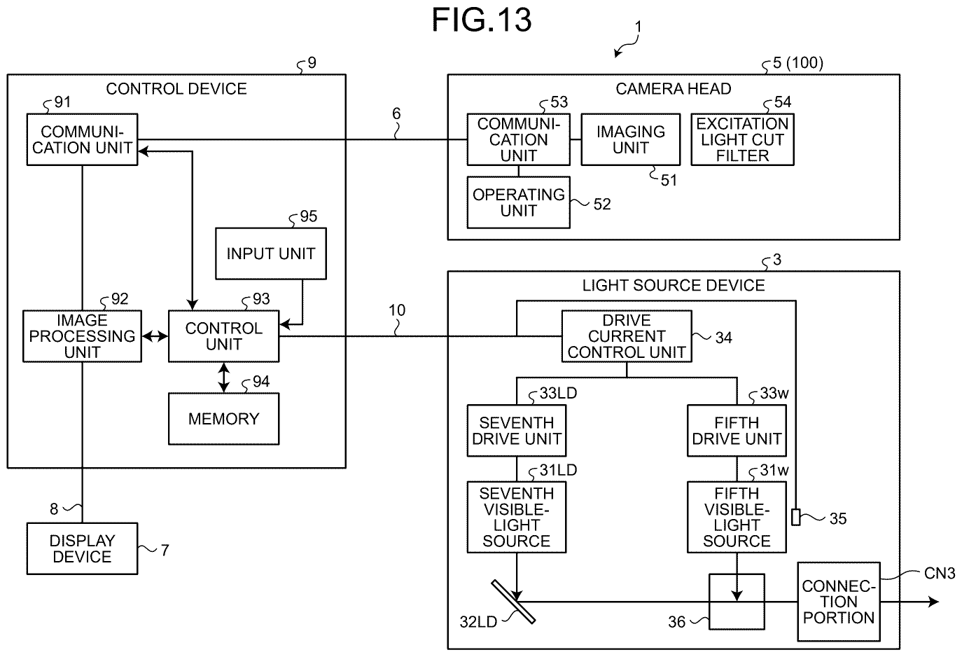

FIG. 13 is a block diagram illustrating the configuration of a main part of the observation system 1 according to the third embodiment.

As illustrated in FIG. 13, the third embodiment is different from the above-described first embodiment in the configuration of a light source device 3 and the configuration of a camera head 5.

As illustrated in FIG. 13, the light source device 3 according to the third embodiment includes a seventh visible-light source 31LD, a multiplexer 36, a seventh dichroic mirror 32LD, and a seventh drive unit 33LD in addition to the fifth visible-light source 31w, the fifth drive unit 33w, the drive current control unit 34, and the temperature measurement unit 35 described above in the second embodiment.

The fifth and seventh visible-light sources 31w and 31LD correspond to a plurality of light sources according to the present disclosure. Then, the fifth and seventh visible-light sources 31w and 31LD emit beams of light in different wavelength bands. The beams of light constitute the illumination light.

Specifically, similarly to the sixth visible-light source 31v described above in the second embodiment, the seventh visible-light source 31LD is provided to adjust the color tone of illumination light emitted from the light source device 3 to the first color tone. That is, the seventh visible-light source 31LD corresponds to the second light source according to the present disclosure. In the third embodiment, the seventh visible-light source 31LD includes a semiconductor laser that emits light (hereinafter, referred to as adjustment light) of a center wavelength: approximately 465 nm between the center wavelength of the blue light BL and the center wavelength of the fluorescent light YL. The seventh visible-light source 31LD emits the adjustment light in a light amount in accordance with supplied drive current.

Note that the seventh visible-light source 31LD may emit not only adjustment light (excitation light) of a center wavelength of approximately 465 nm but adjustment light (excitation light) in another wavelength band as long as the adjustment light (excitation light) has a center wavelength different from the emission spectra of the blue light BL and the fluorescent light YL. For example, the seventh visible-light source 31LD may be adopted. The seventh visible-light source 31LD emits adjustment light (excitation light) in a wavelength band of 430 nm or less, 450 to 500 nm, or approximately 680 to 700 nm. Furthermore, the seventh visible-light source 31LD may include one semiconductor laser or a plurality of semiconductor lasers.

The observation system 1 according to the third embodiment includes a system that enables fluorescence observation. In the fluorescence observation, excitation light is applied to a drug administered into a living body to excite the drug, and fluorescence emitted from the drug is observed. Then, adjustment light emitted from the seventh visible-light source 31LD is excitation light that excites the drug.

The seventh dichroic mirror 32LD reflects adjustment light, and transmits light in a wavelength band different from that of the adjustment light. Note that a mirror that reflects light in substantially all wavelength bands may be adopted instead of the seventh dichroic mirror 32LD.

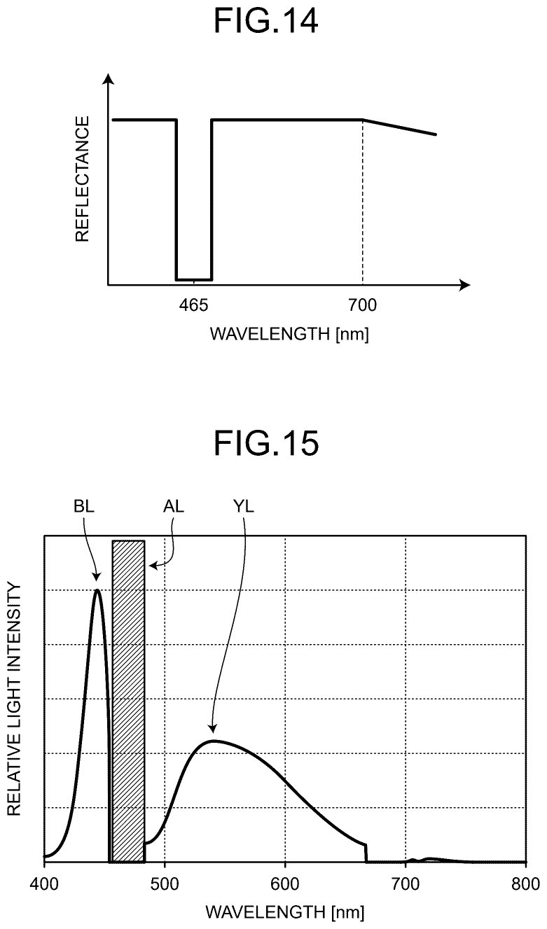

FIG. 14 illustrates the reflection characteristics of the multiplexer 36. Here, in FIG. 14, the horizontal axis represents a wavelength (nm), and the vertical axis represents reflectance. FIG. 15 illustrates the wavelength characteristics of illumination light emitted from the light source device 3. Here, in FIG. 15, the horizontal axis represents a wavelength (nm), and the vertical axis represents relative light intensity.

As illustrated in FIG. 14, the multiplexer 36 includes a band cut filter that transmits adjustment light (center wavelength: approximately 465 nm) and that reflects light in a wavelength band other than that of the adjustment light. Then, the multiplexer 36 transmits adjustment light reflected by the seventh dichroic mirror 32LD, and reflects white light (blue light BL and fluorescent light YL) in the same direction as the traveling direction of the adjustment light. Note that FIG. 15 expresses adjustment light AL reflected by the seventh dichroic mirror 32LD by using a hatched square.

Then, the white light (blue light BL and fluorescent light YL) and the adjustment light AL, whose traveling directions are aligned by the seventh dichroic mirror 32LD and the multiplexer 36, are let in to the incident end of the light guide 4 connected to the connection portion CN3.

Note that the multiplexer 36 is not limited to the above-described band cut filter. A two-branched bundle fiber may be adopted. That is, the multiplexer 36 lets in the adjustment light AL reflected by the seventh dichroic mirror 32LD from one of the two branches of the bundle fiber, and lets in white light (blue light BL and fluorescent light YL) from the other. After multiplexing these beams of light, the multiplexer 36 guides the light obtained by the multiplexing to an incident end of the light guide 4 connected to the connection portion CN3.

The seventh drive unit 33LD supplies drive current designated by the drive current control unit 34 to the seventh visible-light source 31LD to drive the seventh visible-light source 31LD.

The drive current control unit 34 according to the third embodiment causes drive currents to be supplied from the fifth and seventh drive units 33w and 33LD to the fifth and seventh visible-light sources 31w and 31LD under the control of the control unit 93.

As illustrated in FIG. 13, the camera head 5 according to the third embodiment includes an excitation light cut filter 54 in addition to the imaging unit 51, the operating unit 52, and the communication unit 53 described above in the first and second embodiments.

The excitation light cut filter 54 is provided on the front stage side of an optical path of the imaging unit 51. The excitation light cut filter 54 includes a band stop filter that removes a specific wavelength band. Here, the wavelength band cut (removed) by the excitation light cut filter 54 includes at least a part of the wavelength band of the adjustment light AL. That is, the excitation light cut filter 54 cuts (removes) at least a part of the adjustment light AL of illumination light (white light (blue light BL and fluorescent light YL) and adjustment light). The illumination light is applied into a living body, reflected in the living body, and directed to the imaging unit 51. The excitation light cut filter 54 then transmits the other light.

Note that the excitation light cut filter 54 may be provided not only in the camera head 5 but in the insertion portion 2.

In Step S1 according to the third embodiment, the control unit 93 executes the following light source control.

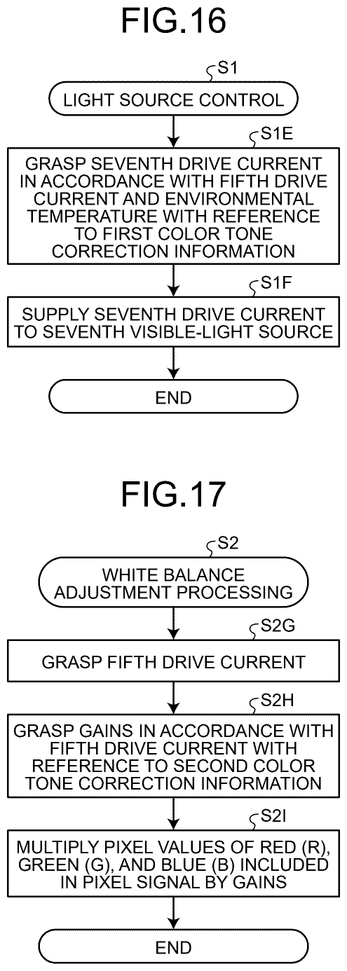

FIG. 16 is a flowchart illustrating Step S1.

Here, the first color tone correction information to be used when the light source control is executed includes a lookup table generated in consideration of the spectral sensitivity of a human eye. In the first color tone correction information, the fifth drive current supplied to the fifth visible-light source 31w and the environmental temperature measured by the temperature measurement unit 35 are associated with seventh drive current. The seventh drive current is supplied to the seventh visible-light source 31LD. When the fifth drive current is supplied to the fifth visible-light source 31w and the fifth visible-light source 31w applies white light into a living body, the seventh drive current corresponding to the fifth drive current in the first color tone correction information is supplied to the seventh visible-light source 31LD, and the seventh visible-light source 31LD applies the adjustment light AL into the living body. The living body thereby has a color suitable for the user such as a surgeon to view an affected area such as a tumor without a feeling of strangeness in a case where the user directly views the inside of the living body with naked eyes. That is, when the fifth and seventh drive currents are respectively supplied to the fifth and seventh visible-light sources 31w and 31LD, the color tone of illumination light to be applied into the living body is adjusted to the first color tone.

Note that the first color tone correction information to be used when the light source control is executed is provided for each luminance of illumination light to be adjusted in dimming control executed by the control unit 93. Here, the seventh drive current in the first color tone correction information corresponding to the luminance of one beam of illumination light is different from the seventh drive current in the first color tone correction information corresponding to the luminance of another beam of illumination light. That is, when the fifth drive current to be supplied to the fifth visible-light source 31w is changed in order to adjust the luminance of illumination light, the seventh drive current is also changed. The color tone of the illumination light is adjusted to the first color tone by changing a light amount of the adjustment light AL.

Then, in Step S1, the control unit 93 executes the light source control with reference to the first color tone correction information stored in the memory 94.

Specifically, the control unit 93 controls the operation of the light source device 3, and supplies the fifth drive current to the fifth visible-light source 31w. The control unit 93 grasps the seventh drive current in accordance with the fifth drive current and the environmental temperature measured by the temperature measurement unit 35 with reference to the first color tone correction information stored in the memory 94 (Step S1E).

After Step S1E, the control unit 93 controls the operation of the light source device 3, and supplies the seventh drive current to the seventh visible-light source 31LD (Step S1F).

Note that, when executing the dimming control, the control unit 93 refers to the first color tone correction information corresponding to the luminance of illumination light to be adjusted under the dimming control in Step S1.

Furthermore, in Step S2 according to the third embodiment, the image processing unit 92 executes the following white balance adjustment processing.

FIG. 17 is a flowchart illustrating Step S2.

Here, the second color tone correction information to be used in the white balance adjustment processing includes a lookup table generated in consideration of the spectral sensitivity of the imaging unit 51 and the display characteristics of the display device 7. In the second color tone correction information, the fifth drive current supplied to the fifth visible-light source 31w and gains associated with each other. Examples of the display characteristics of the display device 7 can include the color balance and a gamma curve of the display device 7. Furthermore, pixel values of red (R), green (G), and blue (B) included in a pixel signal are multiplied by the gains in the white balance adjustment.

As described above, in the third embodiment, the excitation light cut filter 54 is provided in the camera head 5. That is, the imaging unit 51 does not image the adjustment light AL emitted from the seventh visible-light source 31LD. Therefore, the second color tone correction information does not contain the seventh drive current supplied to the seventh visible-light source 31LD as an element for guiding gains to be used in white balance adjustment.

Note that the fifth drive current supplied to the fifth visible-light source 31w correspond to the characteristics of the first light source according to the present disclosure. Then, the above-described second color tone correction information is not required to be generated in consideration of the display characteristics of the display device 7 as long as the second color tone correction information includes a lookup table generated in consideration of the imaging characteristics of the imaging device according to the present disclosure. Furthermore, in the above-described second color tone correction information, information associated with the gains is not limited to the fifth drive current supplied to the fifth visible-light source 31w. A light amount of white light (blue light BL and fluorescent light YL) emitted from the fifth visible-light source 31w or the color tone of illumination light may be adopted as long as the light amount or the color tone corresponds to the characteristics of the first light source according to the present disclosure.

Then, in Step S2, the image processing unit 92 grasps the fifth drive current supplied by the control unit 93 to the fifth visible-light sources 31w in Step S1 (Step S2G).

After Step S2G, the image processing unit 92 grasps gains in accordance with the grasped fifth drive current with reference to the second color tone correction information stored in the memory 94 (Step S2H).

After Step S2H, the image processing unit 92 executes white balance adjustment processing of multiplying the pixel values of the red (R), the green (G), and the blue (B) included in the pixel signal by the grasped gains (Step S2I). This causes the color tone of the captured image to be adjusted to the second color tone.

According to the above-described third embodiment, the following effect is exhibited in addition to effects similar to those in the above-described first and second embodiments.

That is, the excitation light cut filter 54 is provided in the observation system 1 according to the third embodiment. The excitation light cut filter 54 cuts (removes) at least a part of the adjustment light AL of illumination light (white light (blue light BL and fluorescent light YL) and adjustment light AL). The illumination light is applied into a living body, reflected in the living body, and directed to the imaging unit 51. Furthermore, the second color tone correction information does not contain the seventh drive current supplied to the seventh visible-light source 31LD as an element for guiding gains to be used in white balance adjustment. Therefore, no white balance adjustment processing is executed in consideration of the adjustment light AL that is not imaged by the imaging unit 51 by performing the white balance adjustment processing on the pixel signal by using the second color tone correction information. The color tone of a captured image can be favorably adjusted to the second color tone.

### Variation 3

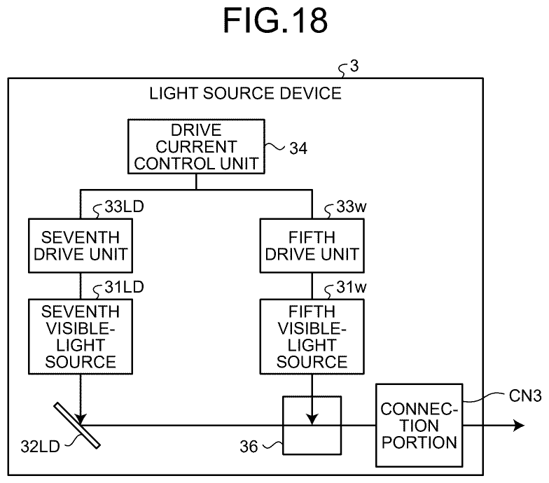

FIG. 18 illustrates Variation 3 of the third embodiment. Specifically, FIG. 18 is a block diagram illustrating the configuration of the light source device 3 according to Variation 3.

Although, in the above-described third embodiment, the first color tone correction information adopts both the fifth drive current and environmental temperature as elements for guiding the seventh drive current, this is not a limitation. Only the fifth drive current may be adopted. That is, in the above-described third embodiment, as in Variation 3 in FIG. 18, the light source device 3 in which the temperature measurement unit 35 is not provided may be adopted.

### Other Embodiments

Although the embodiments for carrying out the present disclosure have been described so far, the present disclosure should not be limited only by the above-described first to third embodiments.

In the above-described first to third embodiments, illumination light constituted of each beam of light emitted from each of the plurality of light sources according to the present disclosure is not limited to white light. The illumination light may be other light as long as the illumination light is visible light. For example, light including no light in one of the red wavelength band, the green wavelength band, and the blue wavelength band may be adopted as illumination light.

Although, in the above-described first to third embodiments, the color tone of the captured image is adjusted to the second color tone by the white balance adjustment processing (Step S2), this is not a limitation.

For example, the color tone of the captured image may be adjusted to the second color tone by appropriately adjusting a color correction matrix. An input matrix is multiplied by the color correction matrix in color correction matrix processing. In the input matrix, pixel values of red (R), green (G), and blue (B) included in the pixel signal are set as matrix elements.

In the above-described first to third embodiments, the second color tone correction information is generated in consideration of the spectral sensitivity of the imaging unit 51. That is, when the camera head 5 having a different imaging unit 51 (imaging unit 51) is used, the second color tone correction information is also required to be changed in accordance with the imaging unit 51. Therefore, for example, a memory that stores identification information for identifying the imaging unit 51 is provided in the camera head 5. Furthermore, the memory 94 stores the second color tone correction information corresponding to the identification information for each piece of identification information. Then, when the camera head 5 is connected to the control device 9, the control unit 93 acquires the identification information from the memory provided in the camera head 5. Furthermore, the image processing unit 92 executes white balance adjustment processing (Step S2) with reference to the second color tone correction information corresponding to the identification information among a plurality of pieces of second color tone correction information stored in the memory 94.

In the above-described first to third embodiments, the second color tone correction information is generated in consideration of the display characteristics of the display device 7. That is, when the display device 7 having different display characteristics is used, the second color tone correction information is also required to be changed in accordance with the display device 7. Therefore, for example, a memory that stores identification information for identifying the display device 7 is provided in the display device 7. Furthermore, the memory 94 stores the second color tone correction information corresponding to the identification information for each piece of identification information. Then, when the display device 7 is connected to the control device 9, the control unit 93 acquires the identification information from the memory provided in the display device 7. Furthermore, the image processing unit 92 executes white balance adjustment processing (Step S2) with reference to the second color tone correction information corresponding to the identification information among a plurality of pieces of second color tone correction information stored in the memory 94.

In the above-described first to third embodiments, the color tone of illumination light and the color tone of a captured image may be adjustable in accordance with an operation of the user such as a surgeon to the operating unit 52 and the input unit 95.

Although, in the above-described first to third embodiments, the observation system 1 is used in the medical field, this is not a limitation. The observation system 1 may be used for observing the inside of a mechanical structure in the industrial field.

Although, in the above-described first to third embodiments, the present disclosure is applied to the observation system 1 using the rigid endoscope (insertion portion 2), this is not a limitation. The present disclosure may be applied to the observation system in Variations 4 and 5 below.

### Variation 4

The observation system according to Variation 4 uses a so-called video scope (flexible endoscope) including an imaging unit on the distal end side of the insertion portion. Hereinafter, for convenience of description, the observation system 1 according to Variation 4 will be referred to as an observation system 1A.

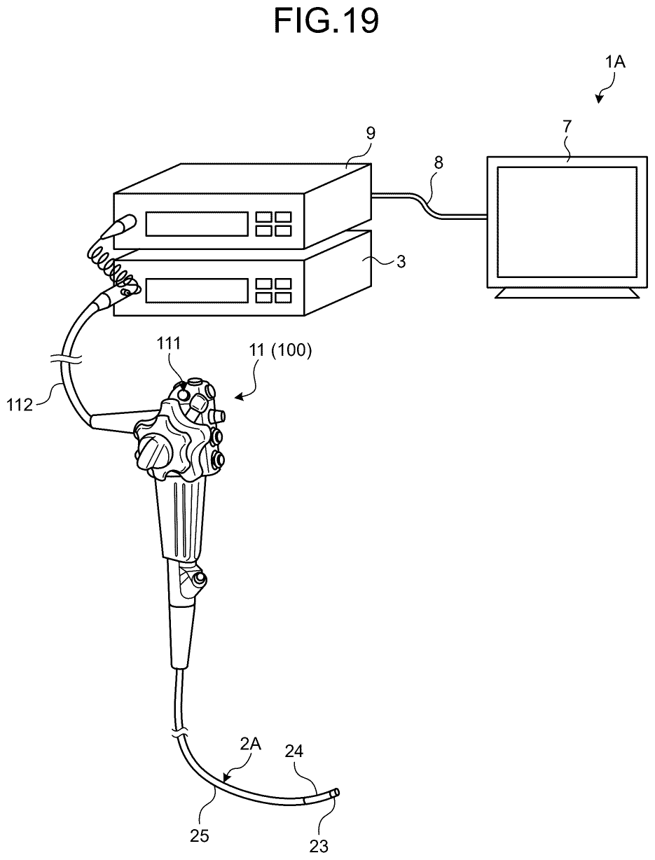

FIG. 19 illustrates Variation 4 of the first to third embodiments.

As illustrated in FIG. 19, the observation system 1A includes an endoscope 11, the light source device 3, the control device 9, and the display device 7. The endoscope 11 captures an inner body image of an observed region by inserting an insertion portion 2A into a living body, and outputs a pixel signal. The light source device 3 generates illumination light emitted from the distal end of the endoscope 11. The control device 9 processes the pixel signal output from the endoscope 11. The display device 7 is connected to the control device 9 via the second transmission cable 8, and displays an image based on a video signal processed by the control device 9.

As illustrated in FIG. 19, the endoscope 11 includes the insertion portion 2A, an operating unit 111, and a universal cord 112. The insertion portion 2A has flexibility and an elongated shape. The operating unit 111 is connected to the proximal end side of the insertion portion 2A, and receives various operations. The universal cord 112 extends from the operating unit 111 in a direction different from the direction in which the insertion portion 2A extends, and incorporates various cables to be connected to the light source device 3 and the control device 9.

As illustrated in FIG. 19, the insertion portion 2A includes a distal end portion 23, a bent portion 24, and a flexible tube portion 25. The bent portion 24 is connected to the proximal end side of the distal end portion 23, includes a plurality of bent pieces, and can be freely bent. The flexible tube portion 25 is connected to the proximal end side of the bent portion 24, and has flexibility and a long shape.

Then, although not specifically illustrated, the distal end portion 23 incorporates a configuration substantially similar to that of the imaging unit 51 described above in the first to third embodiments. Furthermore, although not specifically illustrated, the operating unit 111 incorporates a configuration substantially similar to that of the communication unit 53 described above in the first to third embodiments. Then, the pixel signal obtained by imaging of the distal end portion 23 (imaging unit) is output to the control device 9 via the operating unit 111 and the universal cord 112.

The above-described endoscope 11 corresponds to the imaging device 100 (FIG. 19) according to the present disclosure.

Even when the flexible endoscope (endoscope 11) described above in Variation 4 is used, effects similar to those of the above-described first embodiment are exhibited.

### Variation 5

The observation system according to Variation 5 uses a surgical microscope that enlarges and images a predetermined visual field region inside a subject (inside living body) or on the surface of the subject (on surface of living body). Hereinafter, for convenience of description, the observation system 1 according to Variation 5 will be referred to as an observation system 1B.

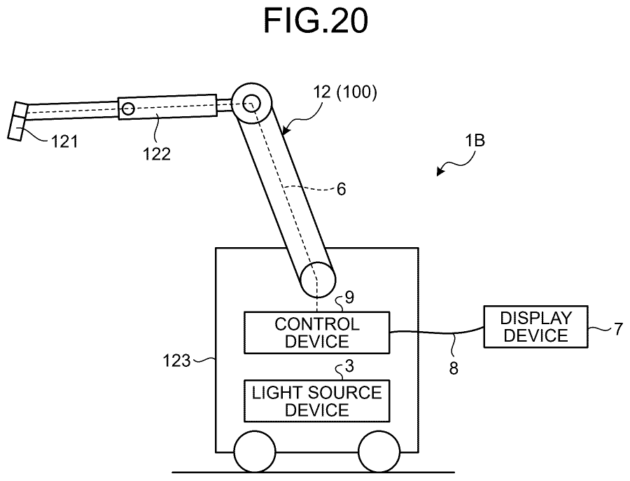

FIG. 20 illustrates Variation 5 of the first to third embodiments.

As illustrated in FIG. 20, the observation system 1B includes a surgical microscope 12, the control device 9, and the display device 7. The surgical microscope 12 captures an image for observing a subject, and outputs a pixel signal. The control device 9 processes the pixel signal output from the surgical microscope 12. The display device 7 is connected to the control device 9 via the second transmission cable 8, and displays an image based on a video signal processed by the control device 9.

The surgical microscope 12 corresponds to an imaging device (FIG. 20) according to the present disclosure. As illustrated in FIG. 20, the surgical microscope 12 includes a microscope portion 121, a support portion 122, and a base portion 123. The microscope portion 121 enlarges and images a minute region of a subject, and outputs a pixel signal. The support portion 122 is connected to a proximal end portion of the microscope portion 121, and includes an arm that rotatably supports the microscope portion 121. The base portion 123 rotatably holds a proximal end portion of the support portion 122, and can move on a floor surface.

Then, as illustrated in FIG. 20, the control device 9 is installed in the base portion 123. Furthermore, although not specifically illustrated, the light source device 3 is also installed in the base portion 123. The light source device 3 generates illumination light to be applied to the subject from the surgical microscope 12.

Note that the base portion 123 may be fixed to a ceiling, a wall surface, or the like to support the support portion 122, instead of being movably provided on the floor surface.

Although not specifically illustrated, the microscope portion 121 incorporates a configuration substantially similar to those of the imaging unit 51 and the communication unit 53 described above in the first to third embodiments. Then, the pixel signal obtained by imaging of the microscope portion 121 (imaging unit) is output to the control device 9 via the first transmission cable 6 arranged along the support portion 122. Furthermore, illumination light emitted from the light source device 3 is applied from the microscope portion 121 toward the subject.

Even when the surgical microscope 12 described above in Variation 5 is used, effects similar to those of the above-described first embodiment are exhibited.

Note that the following configurations also belong to the technical scope of the present disclosure.
(1) An observation system including: a plurality of light sources configured to apply beams of light in different wavelength bands to a subject; an imaging device configured to image the subject to which illumination light including the beams of light from the plurality of light sources is applied; and a control device configured to control operation of at least one of the plurality of light sources, and generate an output image output to a display device by performing image processing on a pixel signal acquired from the imaging device, wherein the control device includes: a light source control unit configured to control operation of at least one of the plurality of light sources, and adjust a color tone of the illumination light to a first color tone; and an image processing unit configured to perform image processing on the pixel signal, and adjust a color tone of the output image to a second color tone such that the color tone of the output image approaches the first color tone.
(2) The observation system according to the above described (1), wherein the light source control unit is configured to control operations of the plurality of light sources, and adjust the color tone of the illumination light to the first color tone by causing beams of light in light amounts in accordance with a light amount ratio of beams of light from the plurality of light sources set based on spectral sensitivity of an eye to be emitted from the plurality of light sources.
(3) The observation system according to the above described (1), wherein the plurality of light sources include: a first light source; and a second light source configured to adjust the color tone of the illumination light, and the light source control unit is configured to adjust the color tone of the illumination light to the first color tone by controlling operation of the second light source based on drive current to be supplied to the first light source.
(4) The observation system according to the above described (3), wherein the plurality of light sources include: a first light source; and a second light source configured to adjust the color tone of the illumination light, the plurality of light sources further include a temperature measurement unit configured to measure environmental temperature around the first light source, and the light source control unit is configured to adjust the color tone of the illumination light to the first color tone by controlling the operation of the second light source based on the environmental temperature.
(5) The observation system according to the above described (3) or (4), wherein the second light source is configured to emit excitation light in accordance with a drug that emits fluorescence when the excitation light is applied.
(6) The observation system according to any one of the above described (1) to (5), wherein the image processing unit is configured to adjust the color tone of the output image to the second color tone by performing image processing on the pixel signal based on imaging characteristics of the imaging device.
(7) The observation system according to the above described (7), wherein the imaging characteristics of the imaging device is spectral sensitivity of the imaging device.
(8) The observation system according to the above described (6) or (7), wherein the image processing unit is configured to adjust the color tone of the output image to the second color tone by performing image processing on the pixel signal based on at least one of a characteristic of at least one of the plurality of light sources and a display characteristic of the display device.
(9) The observation system according to the above described (8), wherein the characteristic of at least one of the plurality of light sources includes drive current supplied to the light source, a light amount of light emitted from the light source, and the color tone of the illumination light.
(10) The observation system according to any one of the above described (6) to (9), wherein the image processing unit is configured to execute white balance adjustment processing of adjusting the color tone of the output image to the second color tone by multiplying at least one of components of beams of light in wavelength bands of red, green, and blue included in the pixel signal by a predetermined gain.
(11) The observation system according to the above described (5), wherein the imaging device includes an imaging unit configured to image the subject to which the illumination light is applied, and an excitation light cut filter configured to block the excitation light among beams of light from the subject, the beams being reflected by the subject and directed to the imaging unit, is provided on a front stage side of an optical path of the imaging unit.
(12) The observation system according to any one of the above described (6) to (11), wherein the image processing unit is configured to adjust the color tone of the output image to the second color tone by performing image processing on the pixel signal based on the characteristics of the first light source excluding the second light source among the plurality of light sources.
(13) The observation system according to any one of the above described (1) to (12), wherein the imaging device includes an endoscope to be inserted into a body of a patient that is the subject.
(14) The observation system according to any one of the above described (1) to (12), wherein the imaging device includes a microscope configured to enlarge and image a predetermined visual field region of an inside or a surface of a body of a patient that is the subject.
(15) The observation system according to any one of the above described (1) to (14), wherein the observation system is used for a medical purpose.
(16) A control device for controlling operation of at least one of a plurality of light sources configured to apply beams of light in different wavelength bands to a subject, and generate an output image output to a display device by performing image processing on a pixel signal acquired from an imaging device configured to image the subject to which illumination light including the beams of light from the plurality of light sources is applied, the control device including: a light source control unit configured to control the operation of at least one of the plurality of light sources, and adjust a color tone of the illumination light to a first color tone; and an image processing unit configured to perform image processing on the pixel signal, and adjust a color tone of the output image to a second color tone such that the color tone of the output image approaches the first color tone.

### REFERENCE SIGNS LIST

1, 1A, 1B OBSERVATION SYSTEM
2, 2A INSERTION PORTION
3 LIGHT SOURCE DEVICE
4 LIGHT GUIDE
5 CAMERA HEAD
6 FIRST TRANSMISSION CABLE
7 DISPLAY DEVICE
8 SECOND TRANSMISSION CABLE
9 CONTROL DEVICE
10 THIRD TRANSMISSION CABLE
11 ENDOSCOPE
12 SURGICAL MICROSCOPE
21 FIBER CONNECTION PORTION
22 EYEPIECE PORTION
23 DISTAL END PORTION
24 BENT PORTION
25 FLEXIBLE TUBE PORTION
31r FIRST VISIBLE-LIGHT SOURCE
31g SECOND VISIBLE-LIGHT SOURCE
31b THIRD VISIBLE-LIGHT SOURCE
31e FOURTH VISIBLE-LIGHT SOURCE
31w FIFTH VISIBLE-LIGHT SOURCE
31v SIXTH VISIBLE-LIGHT SOURCE
31LD SEVENTH VISIBLE-LIGHT SOURCE
32r FIRST DICHROIC MIRROR
32g SECOND DICHROIC MIRROR
32b THIRD DICHROIC MIRROR
32e FOURTH DICHROIC MIRROR
32w FIFTH DICHROIC MIRROR
32v SIXTH DICHROIC MIRROR
32LD SEVENTH DICHROIC MIRROR
33r FIRST DRIVE UNIT
33g SECOND DRIVE UNIT
33b THIRD DRIVE UNIT
33e FOURTH DRIVE UNIT
33w FIFTH DRIVE UNIT
33v SIXTH DRIVE UNIT
33LD SEVENTH DRIVE UNIT
34 DRIVE CURRENT CONTROL UNIT
35 TEMPERATURE MEASUREMENT UNIT
36 MULTIPLEXER
51 IMAGING UNIT
52 OPERATING UNIT
53 COMMUNICATION UNIT
54 EXCITATION LIGHT CUT FILTER
91 COMMUNICATION UNIT
92 IMAGE PROCESSING UNIT
93 CONTROL UNIT
94 MEMORY
95 INPUT UNIT
100 IMAGING DEVICE
111 OPERATING UNIT
112 UNIVERSAL CORD
121 MICROSCOPE PORTION
122 SUPPORT PORTION
123 BASE PORTION
AL ADJUSTMENT LIGHT
BL BLUE LIGHT
CN1, CN2 CONNECTOR
CN3 CONNECTION PORTION
Lr, Lg, Lb CURVE
YL FLUORESCENT LIGHT

1. An observation system comprising:
a plurality of light sources configured to apply beams of light in different wavelength bands to a subject;
an imaging device configured to image the subject to which illumination light including the beams of light from the plurality of light sources is applied; and
a control device configured to
control operation of at least one of the plurality of light sources, and
generate an output image output to a display device by performing image processing on a pixel signal acquired from the imaging device,
wherein the control device includes:
a light source control unit configured to
control operation of at least one of the plurality of light sources, and
adjust a color tone of the illumination light to a first color tone; and
an image processing unit configured to
perform image processing on the pixel signal, and
adjust a color tone of the output image to a second color tone such that the color tone of the output image approaches the first color tone.

2. The observation system according to claim 1, wherein the light source control unit is configured to
control operations of the plurality of light sources, and
adjust the color tone of the illumination light to the first color tone by causing beams of light in light amounts in accordance with a light amount ratio of beams of light from the plurality of light sources set based on spectral sensitivity of an eye to be emitted from the plurality of light sources.

3. The observation system according to claim 1, wherein
the plurality of light sources include:
a first light source; and
a second light source configured to adjust the color tone of the illumination light, and
the light source control unit is configured to adjust the color tone of the illumination light to the first color tone by controlling operation of the second light source based on drive current to be supplied to the first light source.

4. The observation system according to claim 3, wherein
the plurality of light sources include:
a first light source; and
a second light source configured to adjust the color tone of the illumination light,
the plurality of light sources further include a temperature measurement unit configured to measure environmental temperature around the first light source, and
the light source control unit is configured to adjust the color tone of the illumination light to the first color tone by controlling the operation of the second light source based on the environmental temperature.

5. The observation system according to claim 3, wherein the second light source is configured to emit excitation light in accordance with a drug that emits fluorescence when the excitation light is applied.

6. The observation system according to claim 1, wherein the image processing unit is configured to adjust the color tone of the output image to the second color tone by performing image processing on the pixel signal based on imaging characteristics of the imaging device.

7. The observation system according to claim 6, wherein the imaging characteristics of the imaging device is spectral sensitivity of the imaging device.

8. The observation system according to claim 6, wherein the image processing unit is configured to adjust the color tone of the output image to the second color tone by performing image processing on the pixel signal based on at least one of a characteristic of at least one of the plurality of light sources and a display characteristic of the display device.

9. The observation system according to claim 8, wherein the characteristic of at least one of the plurality of light sources includes drive current supplied to the light source, a light amount of light emitted from the light source, and the color tone of the illumination light.

10. The observation system according to claim 6, wherein the image processing unit is configured to execute white balance adjustment processing of adjusting the color tone of the output image to the second color tone by multiplying at least one of components of beams of light in wavelength bands of red, green, and blue included in the pixel signal by a predetermined gain.

11. The observation system according to claim 5, wherein
the imaging device includes an imaging unit configured to image the subject to which the illumination light is applied, and
an excitation light cut filter configured to block the excitation light among beams of light from the subject, the beams being reflected by the subject and directed to the imaging unit, is provided on a front stage side of an optical path of the imaging unit.

12. The observation system according to claim 6, wherein the image processing unit is configured to adjust the color tone of the output image to the second color tone by performing image processing on the pixel signal based on the characteristics of the first light source excluding the second light source among the plurality of light sources.

13. The observation system according to claim 1, wherein the imaging device includes an endoscope to be inserted into a body of a patient that is the subject.

14. The observation system according to claim 1, wherein the imaging device includes a microscope configured to enlarge and image a predetermined visual field region of an inside or a surface of a body of a patient that is the subject.

15. The observation system according to claim 1, wherein the observation system is used for a medical purpose.

16. A control device for controlling operation of at least one of a plurality of light sources configured to apply beams of light in different wavelength bands to a subject, and generate an output image output to a display device by performing image processing on a pixel signal acquired from an imaging device configured to image the subject to which illumination light including the beams of light from the plurality of light sources is applied, the control device comprising:
a light source control unit configured to
control the operation of at least one of the plurality of light sources, and
adjust a color tone of the illumination light to a first color tone; and
an image processing unit configured to
perform image processing on the pixel signal, and
adjust a color tone of the output image to a second color tone such that the color tone of the output image approaches the first color tone.
